(19) United States
(12) Patent Application Publication
(10) Pub. No.: US 2023/0248384 A1
(43) Pub. Date: Aug. 10, 2023

(54) DETACHABLE BLADE TRIGGER

(71) Applicant: Covidien LP, Mansfield, MA (US)

(72) Inventor: Amir Roomi, Boulder, CO (US)

(21) Appl. No.: 17/592,637

(22) Filed: Feb. 4, 2022

(51) Int. Cl. A61B 17/28; A61B 17/29

(52) U.S. Cl. CPC A61B 17/282; A61B 17/29; A61B 2017/2929

(57) ABSTRACT

A detachable trigger assembly for an endoscopic surgical instrument includes a hub having an aperture defined therein adapted to encapsulate a trigger of a surgical instrument upon selective engagement thereof, the trigger configured to advance a knife blade upon actuation thereof. One or more extensions are configured to extend proximally from a side of the hub, each extension including a finger flange depending therefrom configured for actuation by a user. Upon engagement of the hub to encapsulate the trigger of the surgical instrument, the finger flanges are disposed proximally relative to the trigger and wherein actuation of the finger flanges correspondingly actuate the trigger to advance the knife blade of the surgical instrument.

### BACKGROUND

### Technical Field

The present disclosure relates generally to the field of surgical instruments. In particular, the disclosure relates to an endoscopic electrosurgical forceps that is configured to include a detachable blade trigger to accommodate smaller-handed surgeons.

### Background of Related Art

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

Prior to tissue treatment, a surgeon typically manipulates and grasps tissue between opposing jaw members of the forceps using a movable handle. Once grasped, the surgeon energizes the forceps to treat tissue between the jaw members. Once treated the surgeon may opt to actuate a trigger to cut the tissue along the seal. In some instances, the trigger is distally disposed relative to the handle and the surgeon's finger may have difficulty reaching the trigger for actuation. As such, it would be desirous to manufacturer an electrosurgical forceps that includes a selectively detachable trigger extension to facilitate actuation of the trigger.

### SUMMARY

Provided in accordance with the present disclosure is a detachable trigger assembly for an endoscopic surgical instrument which includes a hub having an aperture defined therein adapted to encapsulate a trigger of a surgical instrument upon selective engagement thereof, the trigger configured to advance a knife blade upon actuation thereof. One or more extensions are configured to extend proximally from respective sides of the hub, each extension including a finger flange depending therefrom configured for actuation by a user. Upon engagement of the hub to encapsulate the trigger of the surgical instrument, the finger flanges are disposed proximally relative to the trigger and wherein actuation of the finger flanges correspondingly actuate the trigger to advance the knife blade of the surgical instrument.

In aspects according to the present disclosure, the trigger assembly further includes a spring operably associated with the trigger and configured to return both the finger flange and the trigger back to a pre-actuated position when the finger flange is released.

In aspects according to the present disclosure, the hub includes two opposing extensions extending proximally from either side thereof, each extension including a finger flange depending therefrom configured for actuation by the user.

In aspects according to the present disclosure, the hub includes one or more mechanical interfaces that are configured to operably engage a corresponding mechanical interface associated with the trigger. In other aspects according to the present disclosure, the hub is selectively engaged with the trigger via snap-fit engagement. In still other aspects according to the present disclosure, the snap-fit engagement includes a cantilever snap-fit interface.

In aspects according to the present disclosure, the hub includes two opposing extensions extending proximally from either side thereof, each extension including a finger flange depending therefrom configured for actuation by the user, each extension configured extend transversally relative to the hub to avoid impeding actuation of a handle of the surgeon instrument.

Provided in accordance with additional aspects according to the present disclosure is a detachable trigger assembly for an endoscopic surgical instrument which includes a hub having an aperture defined therein adapted to at least partially encapsulate a trigger of a surgical instrument upon selective engagement thereof on either side of a handle of the surgical instrument to avoid impeding actuation thereof, the trigger configured to advance a knife blade upon actuation thereof. A pair of opposing extensions extend proximally from either side of the hub, each extension including a finger flange depending therefrom configured for actuation by a user. Upon engagement of the hub to encapsulate the trigger of the surgical instrument, the finger flanges are disposed proximally relative to the trigger and wherein actuation of either finger flange correspondingly actuates the trigger to advance the knife blade of the surgical instrument.

In aspects according to the present disclosure, a spring is operably associated with the trigger and configured to return both finger flanges and the trigger back to a pre-actuated position when one finger flange is released.

In aspects according to the present disclosure, the hub includes one or more mechanical interfaces that are configured to operably engage a corresponding mechanical interface associated with the trigger. In other aspects according to the present disclosure, the hub is selectively engaged with the trigger via snap-fit engagement. In still other aspects according to the present disclosure, the snap-fit engagement includes a cantilever snap-fit interface.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIG. 1 is a perspective view of an in-line, electrosurgical forceps according to an embodiment of the present disclosure including a housing, an elongated shaft, an end effector and an actuation handle;

FIG. 2A is an enlarged, perspective view of the end effector of FIG. 1 depicted with a pair of jaw members in an open configuration;

FIG. 2B is an enlarged, perspective view of the end effector of FIG. 1 depicted with the pair of jaw members in a closed configuration;

FIG. 3A is a perspective view of the end effector and elongated shaft of FIG. 1 with parts separated;

FIG. 3B is cross-sectional view taken along line 3B-3B of FIG. 3A showing a distal portion of the electrosurgical forceps of FIG. 1 depicting a tube guide;

FIG. 4 is a perspective view of a proximal portion of the forceps of FIG. 1 with a portion of the housing removed revealing internal components;

FIG. 5 is a partial, side view of a proximal portion of the forceps of FIG. 1;

FIGS. 6A-6B are perspective views of one embodiment of a detachable trigger assembly according to the present disclosure shown engaged atop the electrosurgical forceps;

FIGS. 7A-7B are various enlarged views of the detachable trigger assembly according to the present disclosure; and

FIG. 8 is a bottom, perspective view of a trigger for the electrosurgical forceps having a mechanical interface configured to operably engage the detachable trigger assembly for assembly thereon.

### DETAILED DESCRIPTION

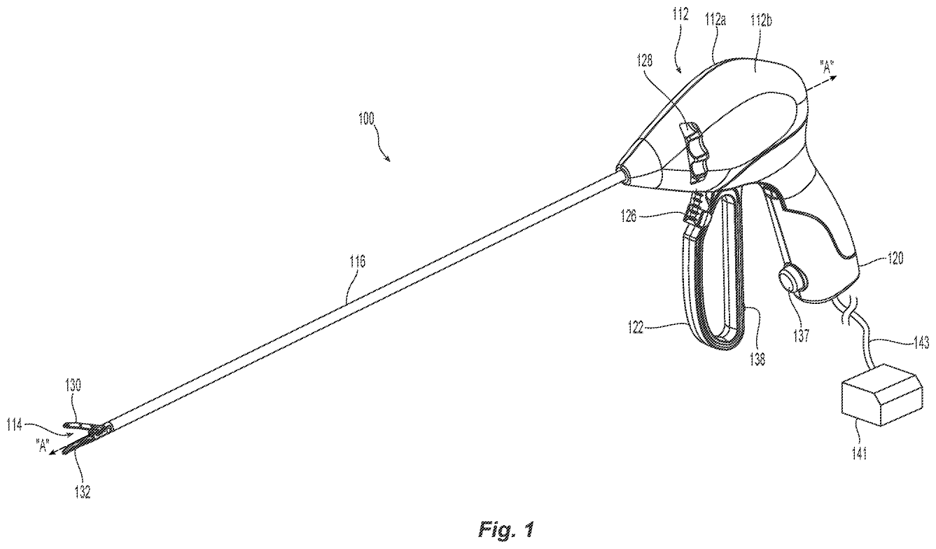

Referring initially to FIG. 1, an electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well. The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

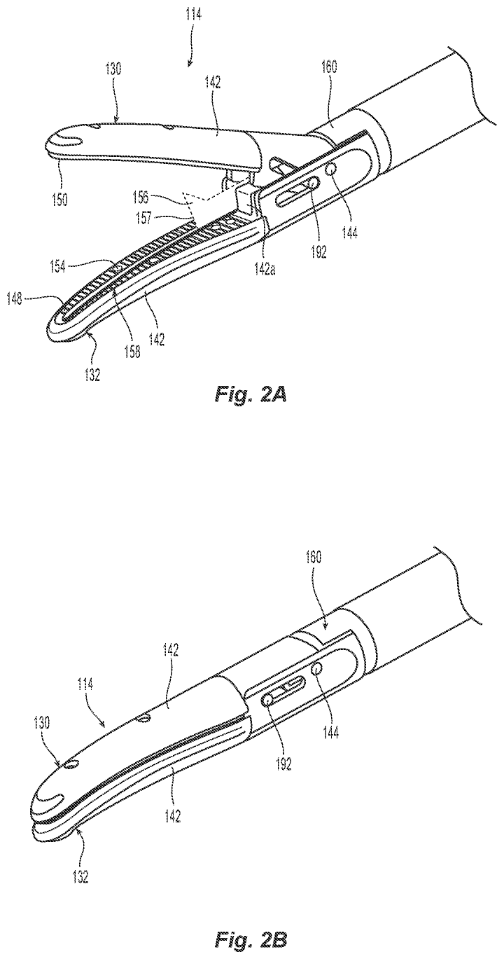

To mechanically control the end effector 114, the housing 112 supports a stationary handle 120, a movable handle 122, a trigger 126 and a rotation knob 128. The movable handle 122 is operable to move the end effector 114 between an open configuration (FIG. 2A) wherein a pair of opposed jaw members 130, 132 are disposed in spaced relation relative to one another, and a closed or clamping configuration (FIG. 2B) wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (see FIGS. 2A and 2B) through the end effector 114 when the end effector 114 is in the closed configuration. The rotation knob 128 serves to rotate the elongated shaft 116 and the end effector 114 about a longitudinal axis A-A extending through the forceps 114.

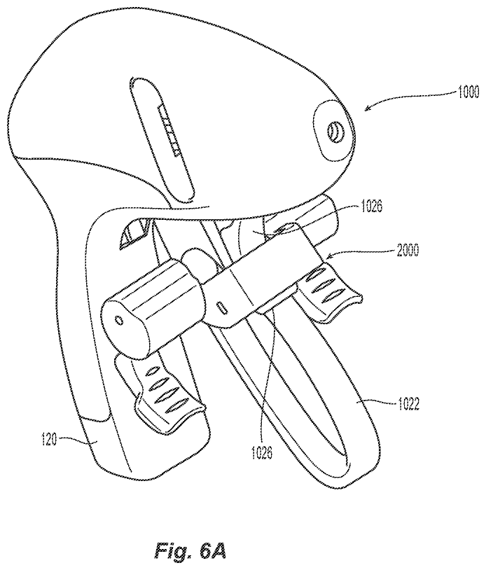

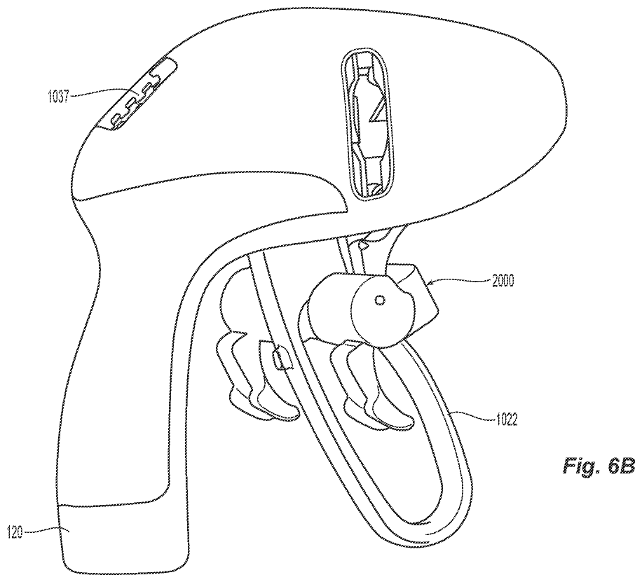

To electrically control the end effector 114, the stationary handle 120 supports a activation switch or depressible button 137 thereon, which is operable by the user to initiate and terminate the delivery of electrosurgical energy to the end effector 114. This particular version of an electrosurgical forceps is a so-called in-line forceps as the activation switch 137 is in-line with the movable handle 122 and the forceps 100 is activated through the stroke of the handle 122. Other, more traditional, electrosurgical forceps 1000 include an activation switch 1037 disposed on the housing (See FIG. 6B).

Turning back to forceps 100, the depressible button 137 is mechanically coupled to a switch (not shown) disposed within the stationary handle 120 and is engageable by a button activation post 138 extending from a proximal side of the moveable handle 122 upon proximal movement of the moveable handle 122 to an actuated or proximal position. The switch is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Sealing Generator and the ForceTriad® Generator sold by Covidien. The cable 143 may include a connector (not shown) thereon such that the forceps 100 may be selectively coupled electrically to the generator 141.

Referring now to FIGS. 2A-2B, the end effector 114 may be moved from the open configuration (FIG. 2A) wherein tissue (not shown) is received between the jaw members 130, 132, and the closed configuration (FIG. 2B), wherein the tissue is clamped and treated. The jaw members 130, 132 pivot about a pivot pin 144 to move the end effector 114 to the closed configuration of FIG. 2B wherein the sealing plates 148, 150 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 (FIG. 2A) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.006 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding. The stop members 154 may define any suitable number, arrangement, and/or configuration, depending on a particular purpose.

The upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (—) terminals associated with the generator 141. Thus, bipolar energy may be provided through the sealing plates 148 and 150 to tissue. Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert 140 and an insulator 142 that serves to electrically insulate the sealing plates 150, 148 from the jaw insert 140 of the jaw members 130, 132, respectively.

Electrosurgical energy may be delivered to the tissue through the electrically conductive seal plates 148, 150 to effect a tissue seal. Once a tissue seal is established, a knife blade 156 having a sharpened distal edge 157 may be advanced through a knife channel 158 defined in one or both jaw members 130, 132 to transect the sealed tissue. Although the knife blade 156 is depicted in FIG. 2A as extending from the elongated shaft 116 when the end effector 114 is in an open configuration, in some embodiments, extension of the knife blade 156 into the knife channel 158 when the end effector 114 is in the open configuration is typically prevented.

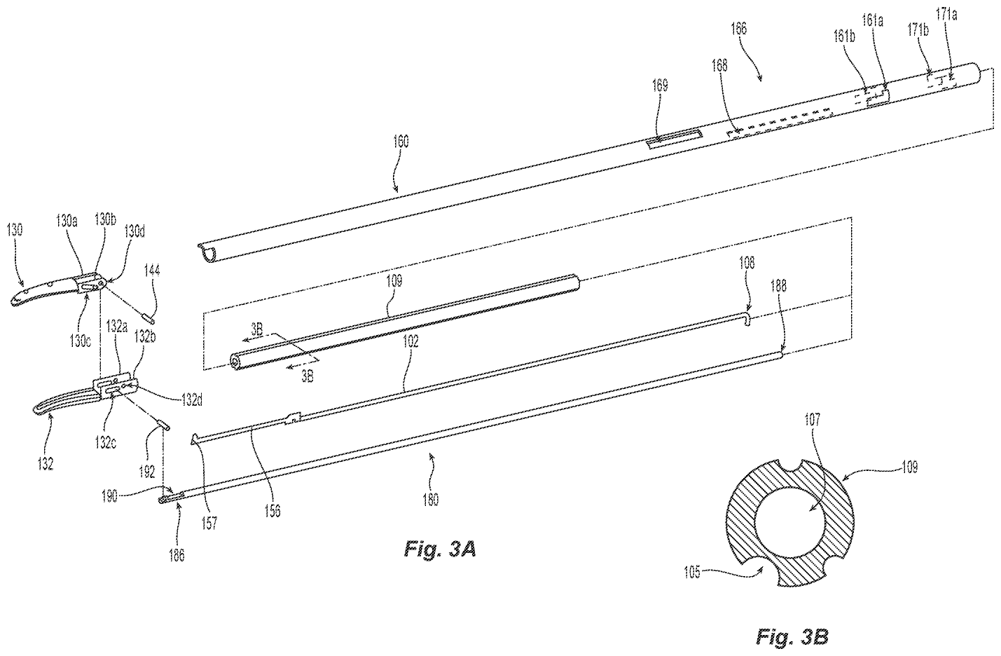

Referring to FIG. 3A, the elongated shaft 116 includes various longitudinal components that operatively couple the end effector 114 to the various actuators supported by the housing 112 (FIG. 1). An outer shaft member 160 defines an exterior surface of the elongated shaft 116 and houses other components therein as described below. The outer shaft member 160 is configured for longitudinal motion with respect to an inner actuation member 180 axially received within the outer shaft member 160. The inner actuation member 180 may be a rod, a shaft, a tube, folded metal, stamped metal, or other suitable structure. A proximal portion 166 of the outer shaft member 160 is configured for receipt within the housing 112 (FIG. 1), and includes features for operatively coupling the outer shaft member 160 to various elements of the housing 112. More specifically, the proximal portion 166 of the outer shaft member 160 includes, in order from distal to proximal, a longitudinal slot 169 to couple the outer shaft member 160 to the rotation knob 128, a longitudinal knife slot 168 defined therethrough, a pair of opposing distal locking slots 161a, 161b, and a pair of opposing proximal locking slots 171a, 171b.

A distal portion 186 of the inner actuation member 180 includes a longitudinal recess 190 defined therein that provides clearance for the pivot pin 144 and thus, permits longitudinal reciprocation of the pivot pin 144 (via longitudinal reciprocation of the outer shaft member 160) independent of the inner actuation member 180. Distally of the longitudinal recess 190, a cam pin 192 is mechanically coupled (e.g., via welding, friction-fit, laser welding, etc) to the distal portion 186 of the inner actuation member 180.

The pivot pin 144 extends through a proximal portion of each of the jaw members 130, 132 to pivotally support the jaw members 130, 132 at the distal end of the inner actuation member 180. A proximal portion of each of the jaw members 130, 132 includes two laterally spaced parallel flanges or “flags” 130a, 130b and 132a, 132b respectively, extending proximally from a distal portion of the jaw members 130 and 132 (FIG. 3A). A lateral cam slot 130c and a lateral pivot bore 130d extend through each of the flags 130a, 130b of the upper jaw member 130 (FIG. 3A). Similarly, a lateral cam slot 132c and a lateral pivot bore 132d extend through each of the flags 132a, 132b of the lower jaw member 132. The pivot bores 130d, 132d receive the pivot pin 144 in a slip-fit relation that permits the jaw members 130, 132 to pivot about the pivot pin 144 to move the end effector 114 between the open and closed configurations (FIGS. 2A and 2B, respectively).

A tube guide 109 is disposed within the outer shaft member 160 and includes a lumen 107 axially disposed therethrough. The inner actuation member 180 is received within the guide lumen 107, which serves to orient and align the inner actuation member 180 within the outer shaft member 160. The knife rod 102 is received within a longitudinal guide recess 105 formed in the outer surface of the guide tube 109. The guide recess 105 serves to guide longitudinal motion of the knife rod 102 within the outer shaft member 160 and to radially space the knife rod 102 from the inner actuation member 180 to prevent the inner actuation member 180 from interfering with reciprocal motion of the knife rod 102.

Rotation knob 128 imparts rotational motion to each of the components of the elongated shaft 116, and to the end effector 114, which is coupled thereto. The rotation knob 128 is supported in the housing 112 and, as shown in FIG. 1, extends radially outward from opposing sides of the housing 112 (only shown extending radially outward from housing half 112b).

End effector 114 is coupled to the distal end of the inner actuation member 180 by the cam pin 192. The cam pin 192 represents a longitudinally stationary reference for longitudinal movement of the outer shaft member 160 and the knife rod 102. The cam pin 192 extends through the flags 132a, 132b of the lower jaw member 132 and the flags 130a and 130b of the upper jaw member 130.

The outer shaft member 160 may be drawn proximally relative to the inner actuation member 180 and the cam pin 192 to move the end effector 114 to the closed configuration (see FIG. 2B). Since the longitudinal position of the cam pin 192 is fixed, and since the cam slot 130c is obliquely arranged with respect to the longitudinal axis A-A, proximal retraction of the outer shaft member 160 induces distal translation of the cam pin 192 through the cam slots 130c, 132c such that the jaw member 130 pivots toward jaw member 132 about the pivot pin 144. Conversely, when the end effector 114 is in the closed configuration, longitudinal translation of the outer shaft member 160 in a distal direction induces proximal translation of the cam pin 192 through the cam slots 130c, 132c such that jaw member 130 pivots away from jaw member 132 toward the open configuration.

The lower jaw member 132 is constructed of three major components: the jaw insert (not shown), the insulator 142, and the sealing plate 148. The flags 132a, 132b of the jaw member 132 define a proximal portion of the jaw insert and a generally u-shaped profile of the jaw insert extends distally to support the tissue engaging portion of the jaw member 132. Upper jaw member 130 includes the same three major components as lower jaw member 132, including sealing plate 150, jaw insert (not shown), and insulator 142, and is constructed in the same manner as lower jaw member 132. However, lower jaw member 132 is fixedly engaged, e.g., welded, to outer shaft member 160, while upper jaw member 130 is pivotable relative to lower jaw member 132 and outer shaft member 160 between the open and closed configurations.

In some embodiments, the insulator 142 on the lower jaw member 132 forms a proximal tissue stop 142a extending therefrom adjacent to the knife channel 158 and proximal to the sealing plate 148. The tissue stop 142a serves to prevent tissue from entering the distal end of the outer shaft member 160 and to prevent splay of the flags 130a, 130b of the upper jaw member 130. In some embodiments, the tissue stop 142a may be formed by the insulator 142 on the upper jaw member 130 or on both the upper jaw member 130 and the lower jaw member 132. The tissue stop 142a may also serve to align the knife blade 156 as the knife blade 156 enters the knife channel 158 defined in the jaw members 130, 132. To this end, the surface of the tissue stop 142a extending along the path of the knife blade 156 may define a chamfered configuration to further facilitate alignment of the knife blade 156 as the knife blade 156 enters the knife channel 158.

The movable handle 122 may be manipulated to impart longitudinal motion to the outer shaft member 160, and the knife trigger 126 may be manipulated to impart longitudinal motion to the knife rod 102. As discussed above, longitudinal motion of the outer shaft member 160 serves to move the end effector 114 between the open configuration of FIG. 2A and the closed configuration of FIG. 2B, and longitudinal motion of the knife rod 102 serves to move knife blade 156 through knife channel 158 (FIG. 2A).

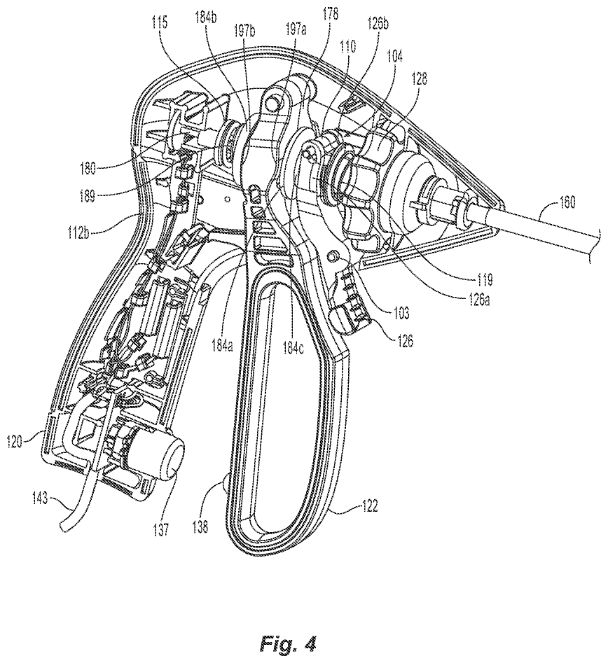

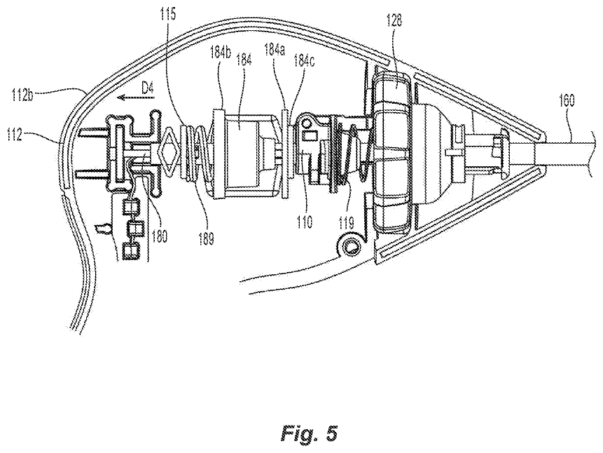

The movable handle 122 is operatively coupled to the outer shaft member 160 by a clevis 178 defined at an upper end of the movable handle 122 (FIG. 4). The clevis 178 is pivotally supported on the housing 112. The clevis 178 extends upwardly about opposing sides of a drive collar 184 (FIG. 5) supported on the outer shaft member 160 and includes rounded drive surfaces 197a and 197b thereon. Drive surface 197a engages a proximal-facing surface of a distal spring washer 184a and drive surface 197b engages a distal facing surface of a proximal rim 184b of the drive collar 184 (FIG. 5). The distal spring washer 184a engages a proximal facing surface of a distal spring stop 184c that, in turn, engages the opposing distal locking slots 161a, 161b (FIG. 3A) extending through the proximal portion 166 (FIG. 3A) of the outer shaft member 160 to couple the distal spring stop 184c to the outer shaft member 160. The drive surfaces 197a, 197b are arranged along the longitudinal axis A-A such that pivotal motion of the movable handle 122 induces corresponding longitudinal motion of the drive collar 184 (FIG. 5) along the longitudinal axis A-A.

Proximal longitudinal motion may be imparted to the outer shaft member 160 by pushing the proximal rim 184b of the drive collar 184 proximally with the movable handle 122 (FIG. 4) as indicated by arrow D4 (FIG. 5). A spring 189 is constrained between a proximal facing surface of the drive collar 184 and a proximal spring stop 115. The proximal spring stop 115 engages the opposing proximal locking slots 171a, 171b (FIG. 3A) extending through the proximal portion 166 (FIG. 3A) of the outer shaft member 160 to couple the proximal spring stop 115 to the outer shaft member 160. Thus, the proximal spring stop 115 serves as a proximal stop against which spring 189 compresses.

Distal longitudinal motion is imparted to the outer shaft member 160 by driving the drive collar 184 distally with the movable handle 122 (FIG. 4). Distal longitudinal motion of the drive collar 184 induces a corresponding distal motion of the outer shaft member 160 by virtue of the coupling of the drive collar 184 to opposing distal locking slots 181a, 181b extending through the proximal portion 166 of the outer shaft member 160 (FIG. 3A).

Proximal longitudinal motion of the outer shaft member 160 draws jaw member 132 proximally such that the cam pin 192 advances distally to pivot jaw member 130 toward jaw member 132 to move the end effector 114 to the closed configuration. Once the jaw members 130 and 132 are closed, the outer shaft member 160 essentially bottoms out (i.e., further proximal movement of the outer shaft member 160 is prohibited since the jaw members 130, 132 contact one another). Further proximal movement of the movable handle 122 (FIG. 4), however, will continue to move the drive collar 184 proximally. This continued proximal movement of the drive collar 184 further compresses the spring 189 to impart additional force to the outer shaft member 160, which results in additional closure force applied to tissue grasped between the jaw members 130, 132 (see FIG. 2B).

Referring again to FIG. 4, the trigger 126 is pivotally supported in the housing 112 about a pivot boss 103 protruding from the trigger 126. The trigger 126 is operatively coupled to the knife rod 102 by a knife connection mechanism 104 such that pivotal motion of the trigger 126 induces longitudinal motion of the knife rod 102. The knife connection mechanism 104 includes upper flanges 126a, 126b of the trigger 126 and a knife collar 110.

As the moveable handle 122 is moved to a fully actuated or proximal position of, the button activation post 138 depresses the depressible button 137, thereby activating the switch disposed within the stationary handle 120 to initiate the delivery of electrosurgical energy to the end effector 114 to generate a tissue seal.

As the movable handle 122 is moved from an intermediate position to a fully actuated or proximal position of, the pressure applied by the jaw members 130, 132 is increased. As the movable handle 122 pivots further the spring 189 is compressed against the proximal spring stop 115, and a tensile force is transmitted through the outer shaft member 160 to the jaw members 130, 132. The tensile force supplied by the spring 189 ensures that the jaw members 130, 132 apply an appropriate pressure to effect a tissue seal.

When the movable handle 122 is in the fully actuated or proximal position, the knife trigger 126 may be selectively moved to a proximal position to advance the knife blade 156 distally through knife channel 158.

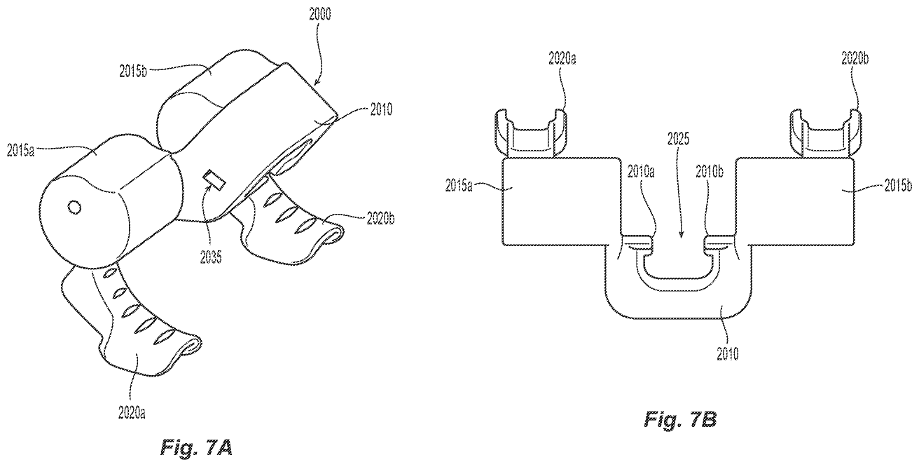

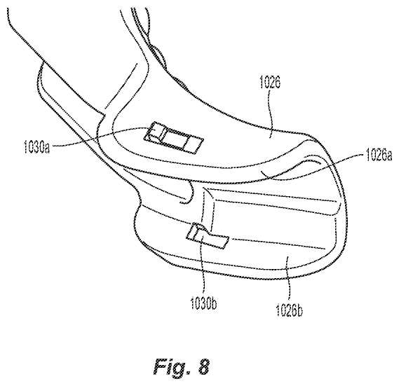

FIGS. 6A-8 show an electrosurgical forceps 1000 for use with sealing or otherwise treating tissue having a selectively detachable trigger assembly 2000 (hereinafter “trigger assembly 2000”) operably engageable therewith. More particularly, trigger assembly 2000 is configured to selectively engage a trigger flange 1026 configured to actuate a knife blade, e.g., knife blade 156, as described in detail above. Trigger assembly 2000 is configured to facilitate actuation by proximally offsetting the point of engagement for a surgeon's finger thereby allowing smaller-handed surgeons more convenient access to advance the knife blade 156.

Trigger assembly 2000 includes a central hub 2010 including an aperture 2025 defined therein configured to operably encase or encapsulate at least a portion of trigger flange 1026 therein. One or more mechanical interfaces, e.g., slots 2035, may be defined in opposing sides of hub 2010 that are configured to operably engage a corresponding set of opposing mechanical interfaces, e.g., cantilever snap-fit interfaces 1030a, 1030b, disposed on the outer peripheral surface of trigger flange 1026 and configured to secure the trigger assembly 2000 to trigger flange 1026. In addition, a pair of inwardly extending protrusions 2010a, 2010b may be configured to engage an underside 1026a, 1026b of the trigger flange 1026 to further secure the trigger assembly 2000 thereto.

Hub 2010 is configured to support opposing extensions on either side thereof, e.g., extensions 2015a, 2015b, that extend transversally relative to handle 1022 of forceps 1000. Each extension 2015a, 2015b supports a respective finger flange, e.g., finger flanges 2020a, 2020b, that each depend therefrom to enable access by the surgeon on either side of the housing 1020 of the forceps 1000 (See FIGS. 6A, 6B). Each finger flange 2020a, 2020b may be shaped similarly to trigger flange 1026 described above or embody a different configuration to facilitate actuation thereof.

As mentioned above, the finger flanges 2020a, 2020b are proximally offset relative to the trigger flange 1026 enabling easier access for smaller-hand surgeon on either side of the housing 1020. Further, the robust nature of the opposing extensions 2015a, 2015b balance the trigger assembly 2000 atop the trigger flange 1026 (e.g., vertically, horizontally and/or rotationally) facilitating easy actuation thereof. Moreover, each opposing extensions 2015a, 2015b is configured extend transversally relative to the hub 2010 a sufficient distance to avoid impeding actuation of the handle 1022 of the forceps 1000 as explained in detail above.

In use, the surgeon simply snaps the trigger assembly 2000 over trigger 1026 and engages the cooperating mechanical interfaces, e.g., slots 2035 and snap-fit cantilevers 1030a, 1030b, to secure the trigger assembly 2000 thereon. Handle 1022 may be moved to actuate the jaw members, e.g., jaw members 130, 132, about tissue as explained above and the forceps 100, 1000 may be energized to treat tissue. The surgeon then actuates either finger flange 2020a or 2020b to advance the knife blade, e.g., knife blade 156, to cut tissue. The return spring, e.g., return spring 119 (FIG. 4), operably associated with trigger flange 1026 to return trigger flange 1026 after release, also cooperates to return the knife blade 156 and finger flange 2020a, 2020b, once released, for subsequent actuation thereof.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

What is claimed is:

1. A detachable trigger assembly for an endoscopic surgical instrument, comprising:
a hub having an aperture defined therein adapted to at least partially encapsulate a trigger of a surgical instrument upon selective engagement thereof, the trigger configured to advance a knife blade upon actuation thereof; and
at least one extension extending proximally from at least one side of the hub, the extension including a finger flange depending therefrom configured for actuation by a user, wherein, upon engagement of the hub to encapsulate the trigger of the surgical instrument, the finger flange is disposed proximally relative to the trigger and wherein actuation of the finger flange correspondingly actuates the trigger to advance the knife blade of the surgical instrument.

2. The detachable trigger assembly for an endoscopic surgical instrument according to claim 1, further comprising a spring operably associated with the trigger and configured to return both the finger flange and the trigger back to a pre-actuated position when the finger flange is released.

3. The detachable trigger assembly for an endoscopic surgical instrument according to claim 1, wherein the hub includes two opposing extensions extending proximally from either side thereof, each extension including a finger flange depending therefrom configured for actuation by the user.

4. The detachable trigger assembly for an endoscopic surgical instrument according to claim 1, wherein the hub includes at least one mechanical interface that is configured to operably engage a corresponding mechanical interface associated with the trigger.

5. The detachable trigger assembly for an endoscopic surgical instrument according to claim 4, wherein the hub is selectively engaged with the trigger via snap-fit engagement.

6. The detachable trigger assembly for an endoscopic surgical instrument according to claim 5, wherein the snap-fit engagement includes a cantilever snap-fit interface.

7. The detachable trigger assembly for an endoscopic surgical instrument according to claim 5, wherein the hub includes two opposing extensions extending proximally from either side thereof, each extension including a finger flange depending therefrom configured for actuation by the user, each extension configured extend transversally relative to the hub to avoid impeding actuation of a handle of the surgeon instrument.

8. A detachable trigger assembly for an endoscopic surgical instrument, comprising:
a hub having an aperture defined therein adapted to at least partially encapsulate a trigger of a surgical instrument upon selective engagement thereof on either side of a handle of the surgical instrument to avoid impeding actuation thereof, the trigger configured to advance a knife blade upon actuation thereof; and
a pair of opposing extensions extending proximally from either side of the hub, each extension including a finger flange depending therefrom configured for actuation by a user, wherein, upon engagement of the hub to encapsulate the trigger of the surgical instrument, the finger flanges are disposed proximally relative to the trigger and wherein actuation of either finger flange correspondingly actuates the trigger to advance the knife blade of the surgical instrument.

9. The detachable trigger assembly for an endoscopic surgical instrument according to claim 8, further comprising a spring operably associated with the trigger and configured to return both finger flanges and the trigger back to a pre-actuated position when one finger flange is released.

10. The detachable trigger assembly for an endoscopic surgical instrument according to claim 1, wherein the hub includes at least one mechanical interface that is configured to operably engage a corresponding mechanical interface associated with the trigger.

11. The detachable trigger assembly for an endoscopic surgical instrument according to claim 10, wherein the hub is selectively engaged with the trigger via snap-fit engagement.

12. The detachable trigger assembly for an endoscopic surgical instrument according to claim 11, wherein the snap-fit engagement includes a cantilever snap-fit interface.
